(19) United States
(12) Patent Application Publication
(10) Pub. No.: US 2023/0410972 A1
(43) Pub. Date: Dec. 21, 2023

(54) SYSTEM AND METHOD FOR REMOTE TREATMENT OF DRUG ADDICTION

(71) Applicant: THE BOARD OF REGENTS OF THE UNIVERSITY OF TEXAS SYSTEM, Austin, TX (US)

(72) Inventors: Michael Alexander Giles, Dallas, TX (US); Madhukar Trivedi, Dallas, TX (US)

(73) Assignee: THE BOARD OF REGENTS OF THE UNIVERSITY OF TEXAS SYSTEM, Austin, TX (US)

(21) Appl. No.: 18/252,152

(22) PCT Filed: Dec. 7, 2021
(86) PCT No.: PCT/US2021/062223

§ 371 Date: May 8, 2023

Related U.S. Application Data
(60) Provisional application No. 63/122,824, filed on Dec. 8, 2020.

(51) Int. Cl. G16H 20/13; G16H 80/00; G16H 40/67; A61J 1/14; A61B 5/00

(52) U.S. Cl. CPC G16H 20/13; G16H 80/00; G16H 40/67; A61J 2200/74; A61B 5/4833; A61J 2200/30; A61J 1/1437

(57) ABSTRACT

A system and method to dispense and monitor a consumable. The system includes a vial configured to transmit at least one characteristic of contents within the vial to a remote location, unlock itself to enable a user to access and remove the contents in response to receipt of an unlock command, and to receive an ingestion confirmation signal when the contents are exposed to body fluids. The method includes determining whether to authorize dispensing of the contents to a patient from the vial, authorizing the dispensing of the contents from the vial, observing, by a clinician over a video conference, dispensing and consumption of the contents by the patient, receiving confirmation from an ingestion sensor that the patient consumed the contents.

### CROSS-REFERENCE TO RELATED APPLICATION

This application claims the benefit of U.S. Provisional Patent Application Ser. No. 63/122,824 filed Dec. 8, 2020, the content of which is incorporated by reference in its entirety.

### ACKNOWLEDGEMENT OF GOVERNMENT SUPPORT

This invention was made with government support under Grant Number MH101078 awarded by the National Institutes of Health (NIH). The government has certain rights in the invention.

### BACKGROUND

### 1. Field of the Invention

Various embodiments of the present inventive concept described herein generally relate to drug treatment such as methadone maintenance therapy (MMT). More specifically, various embodiments of the present inventive concept relate to a combination of a methadone-IEM tablet with a tamper-resistant ingestible event marker (IEM) device, a vial for dispensing the tablet, and an opiate use disorder digital health software platform.

### 2. Discussion of Related Art

Millions of Americans with opioid use disorder (OUD) are unable or unwilling to receive MMT. Currently, MMT protocol requires the patient to visit a licensed methadone treatment program (MTP) daily, for many months so that the methadone consumption can be directly observed and documented by MTP staff. Active employment (or other obligations) and MTP proximity/operating hours thus pose significant barriers to anyone in need of MMT. For example, OUD patients living in rural or medically under-severed areas are typically unable to receive MMT. Moreover, patients who make daily MTP visits often feel stigmatized by this process and will instead opt for effective treatments that do not have this requirement. The COVID-19 pandemic has only amplified the issue of access, which has become problematic even for previously well-served areas due to the necessary, but costly social distancing and shelter-in-place mandates present throughout the country.

To continue delivering care to MTP patients during the COVID-19 pandemic, profound reductions in the stringency of MTP safety standards have temporarily been made. Changes so far have included the removal of the requirement to directly observe medication consumption and an alteration in the frequency of random drug testing. These temporary changes, while helpful for increasing access to MTPs during this pandemic, entail a requisite reduction in patient safety, and therefore do not represent long-term solutions for the millions of patients who previously did not have access to an MTP. Specific safety concerns of these unmonitored take-home methadone regimens include a near-term rise in overdose rates, early refills, and diverted tablets; and to date, over 20 states have reported an increase in opioid-related mortality during the COVID-19 pandemic. A more robust, safe method of providing patients with take-home methadone would not only mitigate the impact of COVID-19 for current MTP patients, it would also address many, if not most, accessibility barriers inherent to the general MTP practice-model.

### SUMMARY

The present inventive concept is an opiate use disorder treatment system and method to provide remotely-monitored methadone with ingestible sensor treatment (R-MIST). R-MIST uses an ingestible sensor and smart-vial technology in conjunction with a single integrated software platform designed for regulatory compliance to treat OUD patients with remotely-monitored methadone maintenance therapy. R-MIST was designed to provide sufferers of OUD with a remotely-monitored methadone treatment regimen that will remedy the geographical, time, productivity, and social constraints inherent to the directly-observed standard. This is accomplished using a combination of technologies and bespoke software, to create the regimen of “H.O.P.E.”:

Handle the medication: methadone is dispensed to the patient in a real-time smart connected medication vial that tracks and records the removal of tagged methadone capsules in real-time and then immediately notifies the physician. This step relieves the patient of the burden of recording the number of doses dispensed.

Observe the consumption: each methadone tablet includes an ingestible sensor, such as by way of non-limiting example encapsulated with an FDA-cleared ingestible sensor from etectRx. When consumed, the ingestible sensor (IS) is activated by the patient stomach acid and transmits a signal to an IS-reader within communication range of the patient, such as worn on a necklace. The captured data is then relayed to a remote database. Once ingestion event information has been stored in the database, it is viewable by authorized clinicians through a web-based clinical dashboard.

Protect the patient: a clinician, such as a physician or nurse, logs into the clinician dashboard at the time the dose is either removed from the smart connected medication vial, reported to be consumed in the mobile application on the patient smartphone, or at the time the patient begins using the IS-reader and waits for the time-stamped ingestion event to become viewable. If the ingestion event has not been uploaded within a certain period of time (e.g., 30 minutes) after the methadone dose was dispensed, a missed detection event (MDE) will be logged into the database. An MDE is of interest, as it may represent any scenario wherein the pill was not consumed (e.g. the pill was lost, diverted, or was not logged due to a technical error). In response to the MDE, the clinician will call the patient to discuss the matter further. At the discretion of the clinician, the smart connected medication vials may be recalled to the clinic so that the remaining doses can be accounted for. Additionally, if the smart connected medication vial detects that multiple, potentially dangerous doses of methadone have been removed, the patient will be instructed by the physician to self-administer naloxone nasal spray (supplied with R-MIST) and EMS services will be called. In the event of a suspected overdose (e.g. multiple doses are removed from the medication vial and the patient is not answering the phone), EMS services will also be called. Finally, the smart-vial technology used in R-MIST includes a locking feature that can be remotely operated by the clinician, and can be programmed to lock/unlock under specific conditions (e.g. in a specific location and/or at specific times). Additional features include, reminders, late-notifications, and location-based notifications. Location-based notifications may be particularly useful for the avoidance relapse triggers (e.g. when a patient nears an area where they used to purchase or use heroin).

Educate the patient: the patient will be invited to attend and participate in remote weekly therapy sessions, for which they may receive an incentive such as a monetary reward (i.e. “contingency management therapy”). The patient will also be able to engage in remote group therapy sessions such as Narcotics Anonymous, directly through the R-MIST platform, and includes positive reinforcement features such as digitally shareable reward tokens for abstinence.

All hardware and software functions needed for the regimen of “H.O.P.E.” may be integrated into a common software environment, but separate patient-facing and physician-facing User Interface (UI) variants can be used. These patient-facing and physician-facing interfaces may access a common cloud database to log and view medication use and compliance data. Text, video, and audio communications between the patient and physician are securely facilitated directly through the software. All data points needed to maintain Substance Abuse and Mental Health Services Administration (SAMHSA) compliance for traditional clinic-based maintenance methadone therapy (such as urine drug testing) are logged into the R-MIST database by the physician and any pertinent data is viewable by the patient through the software.

R-MIST addresses the most common barriers inherent to MMT, thereby making MMT available to millions of additional people. IEM systems are able to reliably and quickly communicate all pertinent medication consumption data (tablet dosage, number of tablets consumed, the time of consumption), to a remote care-provider. The combination eliminates the need for daily MTP visits, yet maintains the strict administration protocols currently required for MMT. The methodology further enhance the efficacy of MMT by incorporating various supportive therapy, safety (e.g. overdose detection and EMS notification), reminders, and positive reinforcement features into the IEM smartphone application.

### BRIEF DESCRIPTION OF DRAWINGS

Various embodiments in accordance with the present disclosure will be described with reference to the drawings, in which:

FIG. 1 shows an embodiment of the present inventive concept;

FIG. 2 shows a flowchart of an embodiment of the present inventive concept;

FIG. 3 shows a vial of an embodiment of the present inventive concept;

FIG. 4 shows a block diagram of electronic components of the vial of FIG. 3; and

FIG. 5 shows a flowchart of safety logic of an embodiment of the present inventive concept.

### DETAILED DESCRIPTION

In the following description, various embodiments will be illustrated by way of example and not by way of limitation in the figures of the accompanying drawings. References to various embodiments in this disclosure are not necessarily to the same embodiment, and such references mean at least one. While specific implementations and other details are discussed, it is to be understood that this is done for illustrative purposes only. A person skilled in the relevant art will recognize that other components and configurations may be used without departing from the scope and spirit of the claimed subject matter.

Specific details are provided in the following description to provide a thorough understanding of embodiments. However, it will be understood by one of ordinary skill in the art that embodiments may be practiced without these specific details. For example, systems may be shown in step diagrams so as not to obscure the embodiments in unnecessary detail. In other instances, processes, structures and techniques may be shown without unnecessary detail in order to avoid obscuring example embodiments.

References to one or an embodiment in the present disclosure can be, but not necessarily are, references to the same embodiment; and, such references mean at least one of the embodiments.

References to any “example” herein (e.g., “for example”, “an example of”, by way of example” or the like) are to be considered non-limiting examples regardless of whether expressly stated or not.

Reference to “one embodiment” or “an embodiment” means that a particular feature, structure, or characteristic described in connection with the embodiment is included in at least one embodiment of the disclosure. The appearances of the phrase “in one embodiment” in various places in the specification are not necessarily all referring to the same embodiment, nor are separate or alternative embodiments mutually exclusive of other embodiments. Moreover, various features are described which may be exhibited by some embodiments and not by others. Similarly, various features are described which may be features for some embodiments but not other embodiments.

The terms used in this specification generally have their ordinary meanings in the art, within the context of the disclosure, and in the specific context where each term is used. Alternative language and synonyms may be used for any one or more of the terms discussed herein, and no special significance should be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only and is not intended to further limit the scope and meaning of the disclosure or of any exemplified term. Likewise, the disclosure is not limited to various embodiments given in this specification.

Without intent to limit the scope of the disclosure, examples of instruments, apparatus, methods and their related results according to the embodiments of the present disclosure are given below. Note that titles or subtitles may be used in the examples for convenience of a reader, which in no way should limit the scope of the disclosure. Unless otherwise defined, technical and scientific terms used herein have the meaning as commonly understood by one of ordinary skill in the art to which this disclosure pertains. In the case of conflict, the present document, including definitions will control.

Several definitions that apply throughout this disclosure will now be presented. The term “substantially” is defined to be essentially conforming to the particular dimension, shape, or other feature that the term modifies, such that the component need not be exact. For example, “substantially cylindrical” means that the object resembles a cylinder, but can have one or more deviations from a true cylinder. The term “comprising” when utilized means “including, but not necessarily limited to”; it indicates open-ended inclusion or membership in the so-described combination, group, series and the like. The term “a” means “one or more” unless the context clearly indicates a single element. The term “about” when used in connection with a numerical value means a variation consistent with the range of error in equipment used to measure the values, for which ±5% may be expected. “First,” “second,” etc., re labels to distinguish components or steps of otherwise similar names, but does not imply any sequence or numerical limitation. “And/or” for two possibilities means either or both of the stated possibilities (“A and/or B” covers A alone, B alone, or both A and B take together), and when present with three or more stated possibilities means any individual possibility alone, all possibilities taken together, or some combination of possibilities that is less than all of the possibilities. The language in the format “at least one of A . . . and N” where A through N are possibilities means “and/or” for the stated possibilities (e.g., at least one A, at least one N, at least one A and at least one N, etc.).

“Clinician” refers to any person authorized to interact with the patient to utilize the one or more embodiments of the present inventive concept to dispense medication. Various laws or regulations may limit who is a qualified clinician for certain types of medications, but that is a legal requirement, not a technical limitation of the present inventive concept. Non-limiting examples of clinicians include physicians, physician assistants, pharmacist, nurses, or interested family members, although the present inventive concept is not limited to any particular person, profession, relationship, or training. In some embodiments, the clinician may be a software programmed to interact with patients and monitor their actions.

“Provider” refers to the entity that employs, supervises, or educated the clinician. Various laws or regulations may limit what is a qualified provider for certain types of medications, but that is a legal requirement, not a technical limitation of the present inventive concept. Non-limiting examples include a hospital, rehab center, pharmacy, doctor's office, although the present inventive concept is not limited to any particular type or qualifications of a provider.

‘Central monitoring location” refers to the provider side operations that execute the various provider side services a discussed herein. It may be a single location, multiple locations operating independently, or a distributed arrangement. Generally, the central monitoring location includes a computer (e.g., a server) having a memory, a processor, a modem, an input/output, and other supporting computer hardware and software. It is foreseen that the central monitoring location may be located in large facilities with distributed computers and human handlers, without deviating from the scope of the present inventive concept. Functionality attributed herein to central monitoring location is preferably implemented by software programmed onto electronic computer hardware. The present inventive concept is not limited to the architecture or layout of the central monitoring location.

“Ingest,” “ingestion” or the like refers to absorption into the body, such as by oral consumption or injection.

When an element is referred to as being “connected,” or “coupled,” to another element, it can be directly connected or coupled to the other element or intervening elements may be present. By contrast, when an element is referred to as being “directly connected,” or “directly coupled,” to another element, there are no intervening elements present. Other words used to describe the relationship between elements should be interpreted in a like fashion (e.g., “between,” versus “directly between,” “adjacent,” versus “directly adjacent,” etc.).

As used herein, the term “front”, “rear”, “left,” “right,” “top” and “bottom” or other terms of direction, orientation, and/or relative position are used for explanation and convenience to refer to certain features of this disclosure. However, these terms are not absolute, and should not be construed as limiting this disclosure.

“Signal” may be defined by its physics (e.g., broadcast frequency) or an identifier code within the broadcast itself. The present inventive concept is not limited to the nature of the signal.

Shapes as described herein are not considered absolute. It is foreseen that one or more surfaces may have waves, protrusions, holes, and/or recesses, etc. to provide rigidity, strength and functionality, without deviating from the scope of the present inventive concept. All recitations of shape (e.g., cylindrical) herein are to be considered modified by “substantially” regardless of whether expressly stated in the disclosure or claims.

It should also be noted that in some alternative implementations, the functions/acts noted may occur out of the order noted in the figures. For example, two steps disclosed or shown in succession may in fact be executed substantially concurrently or may sometimes be executed in the reverse order, depending upon the functionality/acts involved.

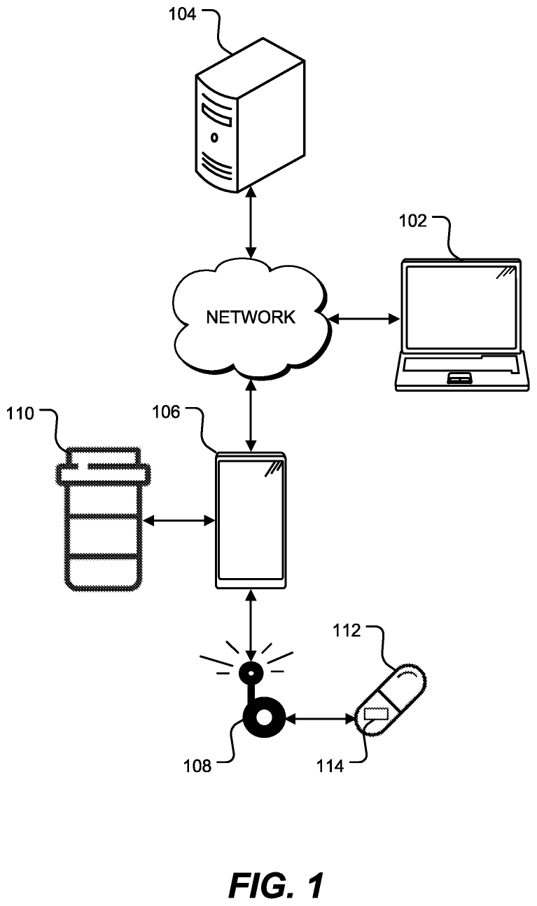

Referring to FIG. 1, R-MIST system 100 includes a clinician side portal 102, a web server 104, a patent side portal 106, sensor reader 108, a vial 110, and consumables 112 in the vial 110. Each of the components is connected by wired or wireless connections as desired or appropriate for their functionality.

Clinician side portal 102 may be any computer or smart device that allows for video conferences and an interface to issue and receive commands. A non-limiting example is a standard internet browser on a computer, although the present inventive concept is not so limited, and any type of computer portal may be used. Client side portal 102 and/or server 104 may serve as part of the central monitoring location.

Patent side portal 106 may be any computer or smart device that allows video conferences and an interface to issue and receive commands. Patient portal 106 may also communicate locally with sensor reader 108, vial 110, and/or sensor 113. A non-limiting example is an app running on a cell phone, although the present inventive concept is not so limited, and any type of computer portal may be used.

Consumable 112 may be a medical product that can be dispensed by and consumed by the patient, either orally or intravenously as dictated by the nature of consumable 112. A non-limiting example is methadone in capsule form, and for purposes of brevity further discussion focuses on the same. However, the present inventive concept is not so limited, and any medication or product, in any form, may be dispensed.

Each dose of consumable 112 may include a sensor 114. Each sensor 114 is configured to detect contact with stomach contents, and to transmit an ingestion confirmation signal in response thereto. The outer shell of consumable 112 protects sensor 115 prior to ingestion, and breaks down when in prolonged contact with body fluids to expose sensor 114. A non-limiting example is consumption and digestion in the presence of stomach acid. When consumable 112 is digested to the point that sensor 114 is exposed to stomach contents, sensor 112 detects the stomach contents and issues a corresponding ingestion confirmation signal.

The ingestion confirmation signal may be universal (no distinction between types of consumables 110 or number of consumables 110) such that the existence of the signal is in and of itself confirmation of ingestion. In the alternative, the signal may be unique to the medication (e.g., methadone pills have one signal, another drug has a different signal), or unique to each pill (e.g., each methadone pill has a unique signal).

Sensor 114, by its nature, may have a limited broadcast range, detection of which may require sensor reader 108 to nearby. Sensor reader 108 may be affixed to a limb of user, such as a bracelet, anklet or necklace (a necklace is shown in FIG. 1). However, the present inventive concept is not so limited, and any form of sensor reader 108 may be used. By way of non-limiting example, sensor reader 108 may be incorporated into patient portal 106.

Sensor reader 108 detects the ingestion confirmation signal from sensor 114 as triggered by digestion. Sensor reader 106 then reports the same to server 104 and/or clinician side portal 102, either directly or through patient side portal 106.

A non-limiting example of a commercial consumable 112 with sensor 114 and sensor reader 108 is the ID-TAG and READER by ETECT-RX. U.S. Pat. No. 9,743,880 also shows a non-limiting example of appropriate components, and is incorporated herein by reference in its entirety.

Vial contains 110 stores a quantity of consumables 112. Vial 110 may include components that control or monitor how many pills are dispensed from vial 110 at any given time. In this embodiment, vial 110 includes a built-in scale, an electronic cap lock that requires an external signal to open, and tamper detection. However, the present inventive concept is not so limited, and some or all of these features may be omitted or replaced with other components.

Vial 110 may be “secure” in the sense that it has some type of electronic lock and/or electronic or manual tamper detection capabilities that are reportable to server 104 and/or clinician side portion 102 for further action. Mechanical impediments, such as child locks that require specific depression, but do not otherwise prevent access or demonstrate tampering, are not “secure” in this context. However, a mechanical impediment that would create visually observable damage to the vial for unauthorized access would qualify.

Communications between some or all of the components described herein may be encrypted, and this may be required under HIPPA regulations, although not a technical requirement of the present inventive concept. However, the present inventive concept is not so limited, and some or all of the communication may be unencrypted.

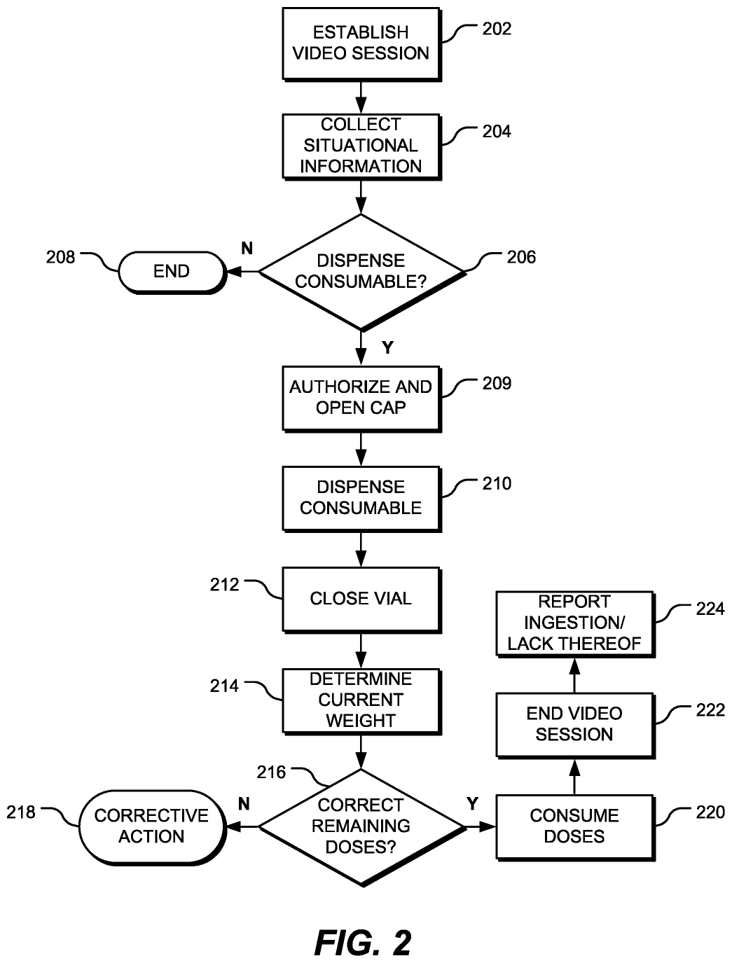

Referring now to FIG. 2, a flowchart 200 of an embodiment for the methodology is shown.

At step 202, a video session is initiated between the patient and the clinician. As discussed in more detail below, this will allow the clinician to visually observe compliance by the patient with the medication distribution protocols. This video session may occur over commercially available video service such as ZOOM, or be a custom solution integrated with other embodiment features. The present inventive concept is not limited to any particular video service or methodology.

At step 204, the system collects information in support of a decision at step 206 whether it is appropriate to dispense consumable 112 to the patient. Non-limiting examples of sources of information include any component shown in FIG. 1. This collection and/or determination may be made at server 114, patient portal 106, clinician side portal 102, or any other location. The present inventive concept is not limited to which components or where the collection and/or determination is made.

The determination at step 206 may be based on a variety of factors from information collected at step 204. Non-limiting examples of factors may include patent history, current date/time, appointment time, location of the patient (as may be provided by patient portal 106 via native location determination capability), current scale reading (are the correct number of pills in vial 110), reported tamper events, accelerometer readings (e.g., is the patient in motion, as may be provided by patient portal 106), open/close status of the cap, and/or charge status of vial 110. However, the present inventive concept is not so limited, and other factors may be included.

The determination at step 206 may be based solely on manual or computer processing of the factors as meeting various criteria. However, the clinician may override the determination based on their expertise and/or authority under law.

The determination at step 206 may decline to dispense medication for a variety of reasons. Non-limiting reasons include, not the appropriate time to dispense, missed prior doses, unknown or restricted location (e.g., a known drug den), vial 110 lacks enough pills for the dose, vial 110 and/or patent portal 106 lacks sufficient change to conduct the process, and/or the cap of vial 110 is already open. However, the present inventive concept is not so limited, and there may be other reasons for declining to dispense consumable 112.

If the decision is made to decline to dispense medication, then the process ends at step 208. This may include a message to the patient, either automatically or orally by the clinician, as to why decision was reached, and/or how the patient can receive their next dosage.

If the decision at step 206 is to dispense consumable 112, then at step 209 authorization is provided allow for the same. The nature of this authorization is based at least in part of the nature of the vial 110. If vial 110 includes a cap with an electronic lock, then the authorization may include the clinician sending an unlock signal to vial 110 (either directly or through patent portal 106) or a code to the patient to enter on vial 110 to unlock vial 110, receipt of which unlocks vial 110 and subsequent can be opened by the patient. If vial 110 has tamper detection, then the authorization may be a note in the patient file that the act of opening the cap (which is physically a tamper event) was authorized. If the vial 110 has inherent dispensing capabilities, then then the authorization may be a command for the dispenser to dispense the pills. If vial 110 just uses a cap with no tamper detection, then authorization would simply be the clinician instructed the patent to open vial 110 under video observation.

At step 210, under video observation by the clinician, the patient removes the approved dose of consumable 112 from vial 110.

At step 212, the patient closes the cap of vial 110 under video supervision by the clinician. If the vial includes an electronic lock or tamper detection, vial 110 can transmit a confirming close signal to server 104 and/or clinician portion 102.

As noted herein, vial 110 may include a scale that measures the weight of pills remaining in vial 110. Since the weight of each individual pill is known to the provider, the number of pills remaining in the vial can be determined from the weight. At step 214, vial 110 transmits information representing the number of consumables 110 remaining in vial 110 post-dispensing. This information may be raw measured data, such as the weight of the pills remaining, or a process of the raw data to identify the specific number of pills remaining. If the patient tried to swap a consumable 112 (e.g., one consumable was authorized, but the patient took two and put a vitamin in as a replacement), then the system could detect the same by the weight differential between consumable 110 and a different sized/density replacement.

At step 216, the system determines whether the number of pills remaining in vial 110 is consistent with expectations. For instance, it is expected the current number of pills in vial 110 should match the number before the cap was unlocked minus the amount of the authorized dose at step 212. If there is a mismatch or some other oddity (e.g., weight shows a pill swap), then at step 218 the system engages in corrective action.

The nature of corrective action is vast, and may depend on the clinician, the provider, some controlling authority (e.g., terms of parole), and/or other requirements of law. By way of example, the clinician may confront the patient and instruct them to return the excess consumables 112 to vial 110. If the patient agrees, then the clinician can unlock vial 110 and recheck the number of consumables per steps 212-216 discussed herein; it would then be up to the clinician whether to take any further action (e.g., alert a parole officer or family member of the infraction, mark the patient for dismissal from the program). If the patient refuses or denies they took the consumables 112, then the clinician can similarly determine whether to take any further action, which may include alerting emergency medical services (EMS) if the clinician has concern that the patient will consume the excess consumables 112 and overdose.

If at step 216 the number of remaining consumables 112 meets expectations, then at step 220 the patient consumes, under video observation, the dispensed consumable 112.

At this point, further video observation may no longer be considered by the clinician to be useful, unless an evaluation is in progress. Accordingly, at step 222 the video call ends.

At step 226, and presuming the patient actually swallowed consumable 110, eventually the sensor reader 108 will detect the ingestion signal from sensor 114 in the ingested consumable 112. Sensor reader 108 reports the same to server 104 and/or patient portion 102 in the manner discussed herein. If the report is not received within an expected period of time, this is =a data point for how the system responds to the next appointment. If the signal was received, then all is well (at least for that step) and the methodology will proceed as normal. If the signal is never received, then that may be a factor under consideration at step 206 during the next appointment as to whether to further dispense consumable 112; by way of non-limiting example, the methodology can refuse to authorize dispensing the next dose.

The methodology discussed herein provides a viable approach to dispensing medication remotely, without forcing patients to come to a specific location. The information from vial 110 confirms the dispensed dosage of consumables 112. The sensors 114 in consumables 112 confirm consumption. Video observation and monitoring from opening of vial 110 through consumption of consumable 112, as advantageously enabled via the present inventive concept, further confirms compliance.

The embodiment shows various steps sequentially, but the present inventive concept is not so limited, and the sequence may be in any order as appropriate. By way of non-limiting example, the dispense decision could be made at 206 before the video is established at 202, thus reducing the burden on clinicians to attend a session when the methodology has already decided that it would not authorize dispensing consumables 112. By way of another example, the patient may jump ahead (authorized or not) and swallow the consumable 112 immediately after dispensing at 210. The end of the video sessions at step 222 may occur after receiving the report of ingestion at step 224. The present inventive concept is not limited to any particular sequence or sub-sequence.

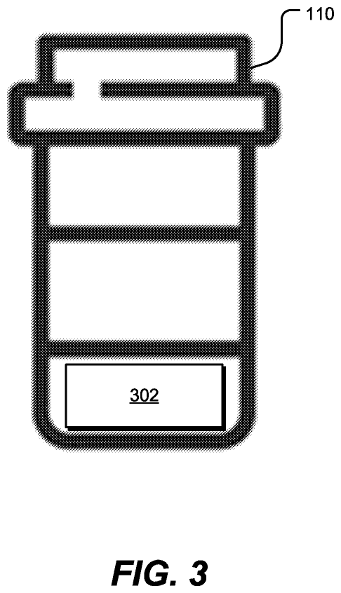

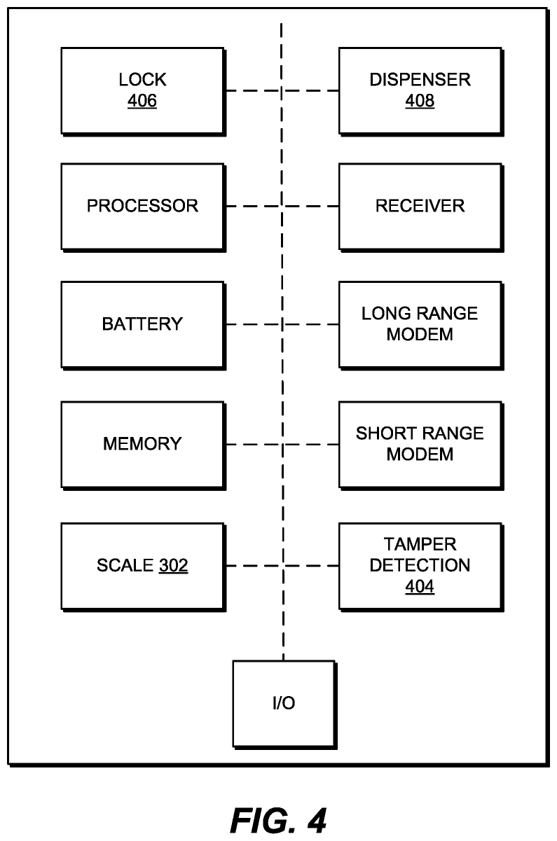

Referring now to FIGS. 3 and 4, an embodiment of vial 110 is shown. Vial 110 may include standard computer components, includes a processor, battery, memory, long range modem (e.g., cellular), short range modem (e.g., BLUETOOTH or Wi-Fi), and a GPS receiver. In addition, vial 110 may include an internal scale 302, tamper detection circuitry 404, a manual or electronic lock 406 that is operable to be converted from a locked configuration, with a cap of the vial 110 unable to be removed, to an unlocked configuration, with the cap of the vial 110 able to be removed, when the authorization signal is received by the electronic lock.

Vial 110 may have a variety of other configurations based on the nature of the security and reminder protocols to access consumables 112. By way of non-limiting examples, vial 110 may itself be a pill dispenser 408 that, in response to the authorization at step 209 releases a predetermined quantity of consumables 112 out of a chute or opening. In such an embodiment, there may be no cap or electronic lock per se, but rather a dispensing mechanism that would receive the authorization code and mechanically dispense the appropriate number of pills in response thereto. In this case, vial 110 may not have or need the scale to measure weight. U.S. Pat. No. 7,359,765 is a non-limiting example of the same, the contents of which are expressly incorporated herein by reference in its entirety.

According to another embodiment of the present inventive concept, vial 110 could be an integrated series of compartments, with an appropriate dose in each compartment and tamper detection circuitry associated with each compartment. U.S. Pat. No. 8,744,620 is a non-limiting example of an integrated series of compartments, the contents of which are expressly incorporated herein by reference in its entirety.

Vial 110, sensor reader 108, patient portal 106 are shown as separated components. However, the present inventive concept is not so limited, and these could be combined. By way of non-limiting example, MEDMINDER combines video communication with pill storage. In another example, patient side portion 106 could be on a cell phone along with an app that acts as sensors reader 108.

Tamper detection for vial 110 may come in a variety of forms. By way of non-limiting example, the interior of vial 110 could include a light sensor that activates in the presence of external light. Another example is an electronic integrity circuit that breaks when the vial is opened. The present inventive concept is not limited to any particular type of tamper detection.

According to another embodiment of the present inventive concept, vial 110 does not utilize an internal scale, instead relying upon an external smart scale that vial 110 rests upon.

The embodiments of the present inventive concept follow the H.O.P.E protocol:

Handle the medication: the patient and caregiver connect with each other via secure video conferencing to facilitate directly observed therapy (DOT). Ingestible sensor (IS) medication capsules are unlocked by the caregiver from the vial. This vial constantly tracks and records the removal of tagged medication capsules in real-time. A safety module such as shown in FIG. 4 decides whether conditions are met to unlock the vial.

Observe the consumption: the IS-methadone capsule is consumed under DOT. The IS is then activated by the user's stomach acid and transmits a signal to the IS-reader. The captured data is then relayed from the reader necklace to the user's smartphone, and then automatically uploaded to the database. Once ingestion event information has been stored in the database as a “positive detection event” (PDE), it becomes immediately viewable by authorized care providers through a web-based “Clinician dashboard”, and the DOT session ends.

Protect the patient: if the ingestion event has not been uploaded within pre-specified time limit from when the dose was dispensed, a missed detection event (MDE) will be logged into the database. An MDE is of interest, as it may represent any scenario wherein the medication was not consumed (e.g., the dose was somehow lost, diverted, or was not logged due to a technical error). In response to the MDE, the care provider will call the participant to discuss the matter further. If the smart-vial detects that multiple, potentially dangerous doses of medication have been removed, the participant will be instructed by the physician to self-administer an antidote (e.g., naloxone nasal spray for R-MIST variants that treat opioid use disorder) and EMS services will be called. At the discretion of the clinician, the vials may be recalled to the clinic so that the remaining doses can be accounted for. Finally, the smart-vial technology used in R-MIST includes a locking feature that can be remotely operated by the clinician when certain criteria met, as determined by the safety module (e.g., in a specific location and/or at specific times). Additional features include reminders, late-notifications, and location-based notifications. Location-based notifications may be particularly useful for the avoidance of relapse triggers (e.g., when a patient nears an area where they used to purchase or use heroin).

Educate the patient: the patient will be invited to attend and participate in remote weekly therapy sessions, for which they will receive a monetary reward (i.e., “contingency management therapy”). The patient will also be able to engage in remote group therapy sessions such as Narcotics Anonymous, directly through the R-MIST platform, and includes positive reinforcement features such as digitally shareable reward tokens for abstinence.

Data points needed to maintain Substance Abuse and Mental Health Services Administration (SAMHSA) compliance for traditional clinic-based maintenance methadone therapy (such as urine drug testing) are logged into the R-MIST database by the physician and any pertinent data is viewable by the patient through the software.

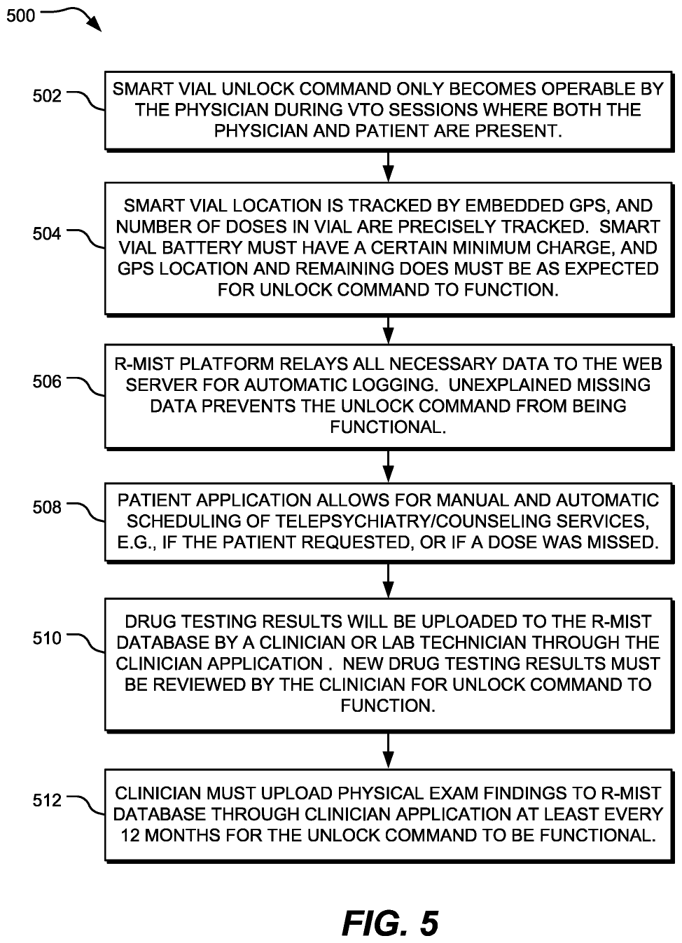

Referring now to FIG. 5, in some instances, the R-MIST workflow includes a method 500 to ensure that methadone is dispensed and consumed in a manner consistent with 2020 U.S. Code of Federal Regulations (C.F.R.), section § 8.12—Federal opioid treatment standards.

For instance, the method 500 can include step 502, in which the smart vial unlock command only becomes operable by the physician during VTO sessions where both the physician and patient are present. Step 502 can correspond to a first 42 C.F.R. § 8.12 standard that requires that methadone consumption must be supervised, which is defined as ensuring it is “administered and dispensed in accordance with its approved product labeling.” The method 500 can include step 504, in which the smart vial location is tracked by embedded GPS, and number of doses in vial are precisely tracked. Smart vial battery must have a certain minimum charge, and GPS location and remaining does must be as expected for unlock command to function. Step 504 can correspond to a second 42 C.F.R. § 8.12 standard that requires diversion control, or preventing tablets from being re-distributed to others. The method 500 can include step 506, in which the R-MIST platform relays all necessary data to the web server for automatic logging. Unexplained missing data prevents the unlock command from being functional. Step 506 can correspond to a third 42 C.F.R. § 8.12 standard that requires recordkeeping, or that OTPS must ensure deviations from “dose, frequency, or the conditions of use described in the approved labeling, are specifically documented.” The method 500 can include step 508, in which the patient application allows for manual and automatic scheduling of telepsychiatry/counseling services, e.g., if the patient requested, or if a dose was missed. Step 508 can correspond to a fourth 42 C.F.R. § 8.12 standard that requires assessment and counseling services, for instance, the physicians must be able to offer counseling services to patients in need. The method 500 can include step 510, in which the drug testing results will be uploaded to the R-MIST database by a clinician or lab technician through the clinician application. New drug testing results must be reviewed by the clinician for unlock command to function. Step 510 can correspond to a fifth 42 C.F.R. § 8.12 standard that requires drug abuse testing services, for instance, that the OTPS must provide “at least eight random drug abuse tests per year.” The method 500 can include step 512, in which clinician must upload physical exam findings to R-MIST database through clinician application at least every 12 months for the unlock command to be functional. Step 512 can correspond to a sixth 42 C.F.R. § 8.12 standard that requires initial medical examination services, or that “OTPs shall require each patient to undergo a complete, fully documented physical evaluation by a program physician or a primary care physician.

However, the present inventive concept is not limited to compliance with those specific standards. It is foreseen that the present inventive concept may be used with other standards, such as standards of other jurisdictions (foreign governments, individual state governments), changing U.S. standards over time, industry standards (e.g., American Medical Association), and/or private standards (e.g., at a particular institution, clinician or the like).

While the embodiments discussed herein are directed to opioid treatment via methadone, the present inventive concept is not so limited. The methodology may be applied to dispensing and/or monitoring of any substance for which a degree of oversight is desired.

Various embodiments discussed or suggested herein can be implemented in a wide variety of operating environments, which in some cases can include one or more user computers, computing devices, or processing devices which can be used to operate any of a number of applications. User or client devices can include any of a number of general purpose individual computers, such as desktop or laptop computers running a standard operating system, as well as cellular, wireless, and handheld devices running mobile software and capable of supporting a number of networking and messaging protocols. Such a system also can include a number of workstations running any of a variety of commercially-available operating systems and other applications for purposes such as development and database management. These devices also can include other electronic devices, such as dummy terminals, thin-clients, gaming systems, and other devices capable of communicating via a network.

Most embodiments utilize at least one network that would be familiar to those skilled in the art for supporting communications using any of a variety of commercially-available protocols, such as TCP/IP, OSI, FTP, UPnP, NFS, CIFS, and AppleTalk. The network can be, for example, a local area network, a wide-area network, a virtual private network, the Internet, an intranet, an extranet, a public switched telephone network, an infrared network, a wireless network, and any combination thereof.

In embodiments where the computing device includes a Web server, the Web server can run any of a variety of server or mid-tier applications, including HTTP servers, FTP servers, CGI servers, data servers, Java servers, and business application servers. The server(s) also may be capable of executing programs or scripts in response requests from user devices, such as by executing one or more Web applications that may be implemented as one or more scripts or programs written in any programming language, such as Java®, C, C #or C++, or any scripting language, such as Perl, Python, or TCL, as well as combinations thereof. The server(s) may also include database servers, including without limitation those commercially available from Oracle®, Microsoft®, Sybase®, and IBM®.

The environment can include a variety of data stores and other memory and storage media as discussed herein. These can reside in a variety of locations, such as on a storage medium local to (and/or resident in) one or more of the computers or remote from any or all of the computers across the network. In a particular set of embodiments, the information may reside in a storage-area network (“SAN”) familiar to those skilled in the art. Similarly, files to enable performance of the functions attributed to the computers, servers, or other network devices may be stored locally and/or remotely, as appropriate. Where a system includes computerized devices, each such device can include hardware elements that may be electrically coupled via a bus, the elements including, for example, at least one central processing unit (CPU), at least one input device (e.g., a mouse, keyboard, controller, touch screen, or keypad), and at least one output device (e.g., a display device, printer, or speaker). Such a system may also include one or more storage devices, such as disk drives, optic storage devices, and solid-state storage devices such as random access memory (“RAM”) or read-only memory (“ROM”), as well as removable media devices, memory cards, flash cards, etc.

Such devices also can include a computer-readable storage media reader, a communications device (e.g., a modem, a network card (wireless or wired), an infrared communication device, etc.), and working memory as described herein. The computer-readable storage media reader can be connected with, or configured to receive, a computer-readable storage medium, representing remote, local, fixed, and/or removable storage devices as well as storage media for temporarily and/or more permanently containing, storing, transmitting, and retrieving computer-readable information. The system and various devices also typically will include a number of software applications, modules, services, or other elements located within at least one working memory device, including an operating system and application programs, such as a client application or Web browser. It should be appreciated that alternate embodiments may have numerous variations from that described herein. For example, customized hardware might also be used and/or particular elements might be implemented in hardware, software (including portable software, such as applets), or both. Further, connection to other computing devices such as network input/output devices may be employed.

Storage media and computer readable media for containing code, or portions of code, can include any appropriate media including storage media and communication media, such as but not limited to volatile and non-volatile, removable and non-removable media implemented in any method or technology for storage and/or transmission of information such as computer readable instructions, data structures, program modules, or other data, including RAM, ROM, EEPROM, flash memory or other memory technology, CD-ROM, digital versatile disk (DVD) or other optic storage, magnetic cassettes, magnetic tape, magnetic disk storage or other magnetic storage devices, or any other medium which can be used to store the desired information and which can be accessed by a system device. Based on the disclosure and teachings provided herein, an individual of ordinary skill in the art will appreciate other ways and/or methods to implement the various embodiments.

The specification and drawings are to be regarded in an illustrative rather than a restrictive sense. It will, however, be evident that various modifications and changes may be made thereunto without departing from the broader spirit and scope of the present inventive concept as set forth in the claims.

What is claimed is:

1. A method to dispense a consumable, the method comprising:
determining whether to authorize remote distribution of a dose of the consumable to a patient from a secure vial, the secure vial containing a quantity of the consumable;
authorizing dispensing of the dose from the secure vial;
observing, by a clinician over a video conference, dispensing and consumption of the dose by the patient; and
receiving confirmation from an ingestion sensor in the dose that the patient consumed the dose.

2. The method of claim 1,
wherein,
the authorizing of the dispensing includes sending an authorization signal to the secure vial.

3. The method of claim 2,
wherein,
the secure vial has an electronic lock, and
the electronic lock is operable to convert from a locked configuration to an unlock configuration when the authorization signal is received by the electronic lock.

4. The method of claim 2,
wherein,
the secure vial is operable to dispense the dose of the consumable when the authorization signal is received by the secure vial.

5. The method of claim 1, further comprising:
measuring a characteristic of the consumable in the secure vial,
wherein the determining declines to authorize remote distribution of the dose in response the characteristics of the consumable in the secure vial being different from an expected characteristic of the consumable.

6. The method of claim 5,
wherein,
the characteristic is a weight of the consumables in the secure vial or a number of consumables in the secure vial.

7. The method of claim 1,
wherein,
the secure vial includes an integrated scale operable to measure the weight of contents in the secure vial,
the method further includes measuring, using the scale, a weight of the consumable in the secure vial, and
the determining declines to authorize remote distribution of the dose in response the measured weight of the consumable in the secure vial, or a number of consumables in the secure vial as determined from the measured weight, being different than a predetermined value.

8. The method of claim 1,
wherein,
the secure vial has tamper detection circuitry, and
the determining results in declining to authorize remote distribution of the dose in response the tamper detection circuitry identifying that the secure vial has been tampered with.

9. The method of claim 1,
wherein,
each consumable has an ingestion sensor, and
the method includes determining, based on receipt of confirmation of ingestion from multiple ingestion sensors, that the patient consumed an amount of consumables in excess of the dose.

10. The method of claim 9, further comprising:
in response to the patient consuming the amount of consumables in excess of the dose:
contacting emergency services;
sending instructions to the patient to administer an antidote; and/or
contacting a contact of the patient.

11. The method of claim 1,
wherein,
each consumable has an ingestion sensor that issues an ingestion confirmation signal triggered by digestion of the consumable, and
the determining results in declining to authorize remote distribution of the dose in response to a failure to receive, for a prior dose of the consumable, the ingestion confirmation signal from the ingestion sensor in the prior dose.

12. A system to dispense a consumable comprising:
a vial with an internal scale and an electronic lock, the vial configured to transmit a weight of contents of the vial and to unlock in response to receipt of an unlock command;
a plurality of consumables, each of the plurality of consumables including an ingestion sensor configured to transmit an ingestion confirmation signal when exposed to body fluids;
a sensor reader configured to receive the ingestion confirmation signal from an ingested one of the plurality of consumables; and
a central monitoring location programmed to perform operations including:
determining whether to unlock the vial based on at least a weight of the contents of the vial and prior receipt of an ingestion confirmation signal from an ingested prior dose of the consumable, and
issuing an unlock command in response to at least a positive result of the determining.

13. The system of claim 12,
wherein,
the determining whether to unlock the vial is further based on whether, after a prior distribution of a dose of the consumable, a weight of the contents of the vial or the number of consumables of the viable correspond to expected amounts.

14. The system of claim 12, further comprising:
a client side portal in communication with the central monitoring location, the sensor reader, and the vial.

15. The system of claim 12,
wherein,
the consumable is an orally consumed medication, and
the ingestion sensor is responsive to a presence of stomach contents.
